(19) United States
(12) Patent Application Publication
(10) Pub. No.: US 2006/0289556 A1
(43) Pub. Date: Dec. 28, 2006

(54) Sheeted cleaning medium and dispenser

(76) Inventor: Gregg R. Rapala, Arlington Heights, IL (US)

Correspondence Address: Gregg R. Rapala, 7 N. Wilshire Lane, Arlington Heights, IL, 60004, US

(21) Appl. No.: 11/452,162

(22) Filed: Jun. 13, 2006

Related U.S. Application Data
(60) Provisional application No. 60/694,040, filed on Jun. 24, 2005.

(51) Int. Cl. A61K 9/70; B65H 1/00

(52) U.S. Cl. 221/63; 424/443

(57) ABSTRACT

An article of a sheeted cleaning medium (such as a tissue, napkin or wipe) and dispenser is provided for easily dispensing a sheeted cleaning medium and, in an embodiment, as a means of storage for the soiled sheeted cleaning medium. The article comprises a sheeted cleaning medium (such as a tissue, napkin or wipe), a member attached to the sheeted cleaning medium, and a typically hand sized or pocket sized container.

This application claims the benefit of Provisional Patent Application Ser. No. 60/694,040 filed Jun. 24, 2005 by the present inventor, Gregg R. Rapala.

### FIELD OF THE INVENTION

This invention relates to sheeted cleaning media (such as facial tissues, napkins and wipes) and a dispenser therefore; and in particular, individually packaged sheeted cleaning media, preferably pocket sized.

### BACKGROUND OF THE INVENTION

This invention relates to sheeted cleaning media using the broadest classifications for such. Such media includes facial tissues, napkins and all types of wipes—for general purposes and specific purposes. The media can be dry, moist or wet; and it can be made of natural, synthetic or compositions of natural and synthetic materials. The invention is anticipated to have wide use as pocket facial tissues, wipes, moist wipes, and wet wipes; and embodiments of these will be described in more detail. The word “tissue” will be used to mean a sheeted cleaning medium, using the broadest classifications for such, unless it is further described (for example, “facial tissue”), or used in the context of what is being described.

Generally, sheeted cleaning media have been packaged in multi-packs, partially for a manufacturer's practicality and economics. The only known exceptions to this are individually packaged wet wipes and medical gauze. The handkerchief is a single sheeted cleaning medium, and had been widely used prior to the appearance of facial tissues. The handkerchief is usually made of cloth and is nicely sized for facial use, but provides no container for storage (either for clean or soiled conditions). However, handkerchiefs are large so a soiled portion can be wrapped within clean sections of the cloth, thereby allowing the handkerchief to be returned to ones pocket without worry of soil transfer to the pocket. Rather than being disposed of like current facial tissues, it's to be laundered after use. The popularity of the handkerchief has diminished over the years, as facial tissues have offered an inexpensive and disposable alternative.

Pocket packs of facial tissues were made for people to carry with them, and to take along when away from home or office. However, facial tissues are much smaller in size than the handkerchief, and therefore do not have the same quantity of material for use, or for wrap-up protection for re-pocketing after use. This often causes more than one tissue to be used. The packages are also bulky to carry, and provide no storage space for the used tissue. Therefore, people do not often carry pocket facial tissue multi-packs, as they are rather large for a pocket and may be needed only on infrequent occasions. Hence, they often fail the real need. Often, loose tissues are carried for convenience instead. Paper napkins are also carried as a substitute for facial tissues, and as a general-purpose wipe. In these instances again, there is no storage means for the clean or used tissue. Dirt, dust and dyes can accumulate on tissues while pocketed, and fibers from the tissue can become loosened and dislodged. Re-pocketing after use may cause more severe material transfers, and often from the soiled tissue fibers to ones pocket.

The only known facial tissue products on the market today are multi-packs. The pocket facial tissue multi-packs typically contain 15 or more tissues. One of the most common and smaller packages is approximately 1″ thick, 2½″ wide, and 4½″ long. The Kleenex® brand by Kimberly-Clark Corporation (Neenah, Wis.) is possibly the most widely used product of this type. These packages supply more than an adequate quantity of tissues. When pocket tissue multi-packs are used, a tissue is removed from a thin plastic film container (or, over-wrap) for use. The tissues are folded into rectangles and are dispensed from the container by lifting an edge of a tissue that lays flat against a folded side of the tissue from within the container; or by removing the entire tissue from the container first. If one is able to grasp and pull the edge that lays flat against the tissue, the tissue may open up pretty much on it's own when slightly shaken. However, often times the entire tissue is removed first, and one then needs to unfold it about two times in one direction and another two times in a different direction to arrive at the full-sized tissue. The Puffs to Go® brand of tissue by Puffs (Dist. by Procter & Gamble, Cincinnati, Ohio) has a tissue that will open up pretty much on its own when removed from the package, with a good hand position on the tissue for use. It is folded similar to the Kleenex® brand with one less fold and has a grasping edge that is positioned differently in the container, but also lays flat against the tissue. The package is also larger, approximately 1″ thick, 4″ wide and 5″ long. This is a large package to carry for occasional tissue use, and is basically a full hand-size to hold. Often, these multi-packs are not even carried on an individual, but rather are placed in a car, travel bag, or large purse. Typically then, the multi-pack cannot be held comfortably while a tissue is being used, and is put down or placed back in ones pocket, purse, bag, or car before a tissue is used. Therefore, upon removal and use of a tissue, one has both the multi-pack and the soiled tissue to re-pocket or re-store. That's now two items, to handle and carry. Also, the items are usually separated from each other by using different pockets, or sections of one's purse, bag, or car. After half or more of the multi-pack's tissues have been used, handling of the package becomes increasingly awkward as the tissues become loose in an increasingly oversized package. It's most awkward with the last few remaining tissues.

Several wet wipe and moist wipe products use plastic film containers that can be resealed. These typically hold about 10 to 30 wipes. The smallest known container for such wet wipe products (multi-pack form) approximates the size of a pocket facial tissue multi-pack, as previously described. These packages have similar drawbacks to the pocket facial tissue packs, but also often dry out before all the wipes are used.

The current availability of individually packaged tissue is extremely limited. No individually packaged consumer facial tissue or dry wipe is known to be available. The closest known product is sterile gauze used in medical applications. In this instance, the sterile gauze is often contained between two flat sheets of a medical grade paper, or a medical grade paper-like plastic such as Tyvek® brand of spun-bonded olefin made by DuPont (Wilmington, Del.).

Individually packaged wet wipes are available. However, these wipes are uncomfortably sized and folded, and are sandwiched between two sheets of a laminate of metal foil and plastic film. The package must be torn open to arrive at the wipe. The wipe, which had been folded into a small rectangle, must then be unfolded. One of the more popular products of this type is the Wet Ones® brand of “Singles” moist wipes, distributed by Playtex Products, Inc. (Dover, Del.). The package is designed for tearing open at a small cut that has been placed into an edge approximately 40% of the way in. One must then grab the folded wipe and remove it from the package. Before explaining the unfolding of the Wet Ones® wipe, it's perhaps best to describe how it was folded. A rectangular wipe is folded back and forth (zigzag) along its width and flattened so that the wipe is approximately a fifth of the original width. The wipe is now the same length, but narrower. Next, this long folded wipe is folded in the middle to make it half as long, and then folded approximately in half again to make it about a quarter of the original length. The result is a rectangular shaped folded wipe approximately one-fifth the original width and about one-fourth the original length of the wipe. To unfold, one opens two folds to arrive at the full length of the wipe, and then picks the edges from the sides to pull open the width. If the outermost edges of the wipe had not been picked and pulled, then only a section of the wipe had been unfolded and it becomes necessary to unfold or pick at these edges again. As these edges lay flat against a folded side of the moist wipe, they can be difficult to peel up, grasp and use to pull open the wipe. As this folded wipe is fairly long, one then typically needs to assist the unfolding of the other portions of the wipe by using ones other fingers as well, or by grasping and pulling the wipe in a different location. All of this, to open and use a wipe, requires a bit of effort.

What is common with all the aforementioned products is that the edge of the tissue that one needs to pull to remove or to unfold the tissue, lays flat against a folded side of the tissue making it difficult to grasp and unfold. Moist wipes and wet wipes are especially difficult to open.

In the selection of pocket facial tissues and wipes, size, convenience, and ease of use are all extremely important. It should fit easily within a pocket, while leaving room for other things. It should be easy to use, with a tissue that quickly and easily dispenses from a container and unfolds by itself, or almost by itself. It should be new and clean, and in a standardized form. After the tissue has been soiled, there should be somewhere to store it, as a means of disposal may not be nearby. It should also be inexpensive, and an item that can be readily asked for and exchanged in public when needed. This invention describes such an article and method. Accordingly, several objects and advantages of the present invention are:

a.) to provide an article that is much smaller than current pocket multi-tissue packs as it contains only a single tissue. Thus, one or more of these items can be easily carried on an individual. This is especially convenient for social events, outdoor activities, and public places such as churches, libraries, and waiting areas.

b.) to provide an article that can be displayed for complimentary use at establishments, similar to displays of business cards, matches, toothpicks, and packets of condiments. Several examples of these establishments might include restaurants, hotels, and funeral homes.

c.) to provide an article that upon which advertisement such as company names and logos, and artistic designs may be printed or placed for promotional or fanciful purposes. Several promotional uses may include gift packages, trade show giveaways, and complementary items at establishments and sponsored events.

d.) to provide an article and method for more quickly removing a tissue from a closed container. Unlike prior art, embodiments of the present invention allow for a tissue to be removed from a container without a separate step of opening the initial closed container.

e.) to provide an article and method for a tissue unfolding on its own or with only minimal assistance.

f.) to provide an article and method for a tissue to dispense close to the hand position normally used during tissue use, so that repositioning of the tissue before use is minimized.

g.) to provide an article and method for a small, thin, and flexible container to be brought up to ones face along with a tissue, during facial tissue use. This allows for more immediate use of a tissue with fewer handling steps.

h.) to provide an article and method that offers a solution to the unsolved need of “Where to put the soiled tissue?” by providing a means of storage for the used tissue, and thus providing for more sanitary handling prior to final disposal. Additionally, the container can be made to both close and re-seal, so as to fully confine the used tissue.

i.) to provide an article and method for simultaneously reducing the bulk and handling steps (for use) of a pocket tissue product.

j.) to provide an article that is an inexpensive and disposable alternative to the handkerchief, with additional enhanced features, while maintaining a similar or more reduced size.

k.) to provide an article that makes exchanging a tissue in public more common and acceptable. One would feel more comfortable requesting a tissue in public, as one would be asking for a standardized item with known quality and sanitary condition. Likewise, a provider of the article will also feel more comfortable in providing a tissue, as this is now in a known form, and is both useful to others and of little expense. The article may also provide the recipient with a means of storage for the used tissue, which is especially useful, as often in public a means of disposal is not readily available.

Further objects and advantages of this invention will become apparent from a consideration of the drawings and ensuing description.

### SUMMARY OF THE INVENTION

The present invention provides a sheeted cleaning medium (such as a facial tissue, napkin or wipe) and typical dispenser for easily dispensing the sheeted cleaning medium and, in an embodiment, a means of storage for the soiled sheeted cleaning medium. The article comprises a sheeted cleaning medium (such as a tissue, napkin or wipe), a member attached to the sheeted cleaning medium, and a typically hand-sized or pocket-sized container. Advertising and/or logos may be applied to the container surface for promotional purposes. The member is preferably both internally and externally exposed from the container and provides a means of opening the container and dispensing the sheeted cleaning medium. The sheeted cleaning medium may be dry, moist or wet; and in embodiments may unfold upon dispensing or with minimal assistance such as a shake of the hand, or by way of pulling a protruding area of the folded sheeted cleaning medium.

### BRIEF DESCRIPTION OF THE DRAWINGS

FIG. 1 is a plan view of a backside of an embodiment of the invention displayed in the palm of a hand.

FIG. 2a is a plan view of a backside of the container of FIG. 1 open and without a tissue assembly.

FIG. 2b is a plan view of a backside of the container of FIG. 2a open and with a tissue assembly partially inserted.

FIG. 2c is a plan view of a backside of the container of FIG. 2b with a tissue assembly fully inserted and lid closed.

FIG. 2d is a plan view of a front side of the container of FIG. 2c on which surface company names and logos can be printed or placed.

FIG. 3a is a plan view of the tissue of FIG. 2b prior to folding, and depicting typical fold lines.

FIG. 3b is a perspective view of the tissue of FIG. 3a depicting a typical first folding of the tissue.

FIG. 3c is a perspective view of the tissue of FIG. 3b depicting a typical secondary folding of the tissue.

FIG. 3d is a perspective view of the tissue of FIG. 3c depicting a typical fully folded tissue and a typical placement of an attached member. The tissue assembly, comprising the tissue and member, is ready to be inserted in a container.

FIG. 4 is a perspective view of the container and tissue assembly of FIG. 2c depicting the dispensing of the tissue, where the tissue begins unfolding as it is being removed from the container.

FIG. 5 is a perspective view of the tissue assembly of FIG. 4 after it has been removed from the container and is unfolding.

FIG. 6 is a perspective view of the tissue assembly of FIG. 5 as the tissue is grasped at its nearest edge from the member, by the hand used to pull the member to dispense the tissue. FIG. 7 is a perspective view of the container of FIG. 4 depicting a typical holding of the lid portion of the container for the purpose of re-inserting the tissue assembly after use.

FIG. 8 is a perspective view of the container of FIG. 7 depicting another typical holding position of the container for the purpose of tissue assembly re-insertion. Often, one can start the tissue assembly re-insertion as depicted in FIG. 7 and then complete it as depicted in FIG. 8.

FIG. 9 is a perspective view depicting a fully re-inserted used tissue assembly within the closed and re-sealed container.

FIG. 10a is a plan view depicting a single piece member/container package.

FIG. 10b is a perspective view of the member/container package of FIG. 1a depicting the insertion of a folded tissue, and with member attachment to the folded tissue.

FIG. 10c is a plan view of the assembly of FIG. 10b with the folded tissue fully inserted and the container closed.

FIG. 11a is a perspective view of an embodiment of a member attached to a wipe and showing typical fold lines.

FIG. 11b is a perspective view of the wipe assembly of FIG. 11a depicting a typical first folding of the wipe.

FIG. 11c is a perspective view of the wipe assembly of FIG. 11b showing the folding more fully compressed.

FIG. 11d is a perspective view of the wipe assembly of FIG. 11c depicting a typical second folding of the wipe.

FIG. 11e is a perspective view of the wipe assembly of FIG. 11d partially inserted in a container.

FIG. 12a is a plan view of an alternate embodiment of the present invention depicting a wipe assembly sandwiched between two sheets of packaging film.

FIG. 12b is a plan view of an alternate embodiment of FIG. 12a with a portion of a member within the construction of the container.

### DETAILED DESCRIPTION OF THE PRESENTLY PREFERRED EMBODIMENTS

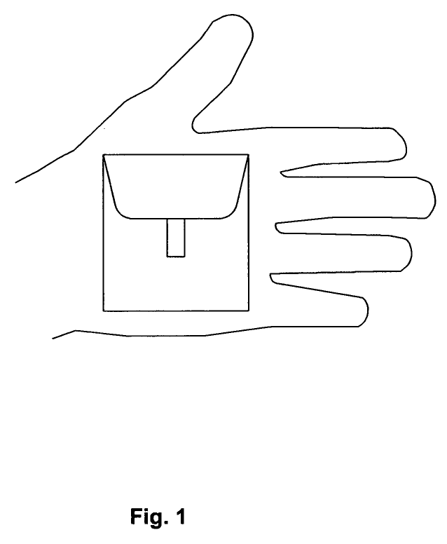

FIG. 1 is a plan view of a backside of an embodiment of the invention displayed in the palm of a hand. This drawing is intended to give the reader a quick overall perspective of a presently preferred embodiment of the present invention.

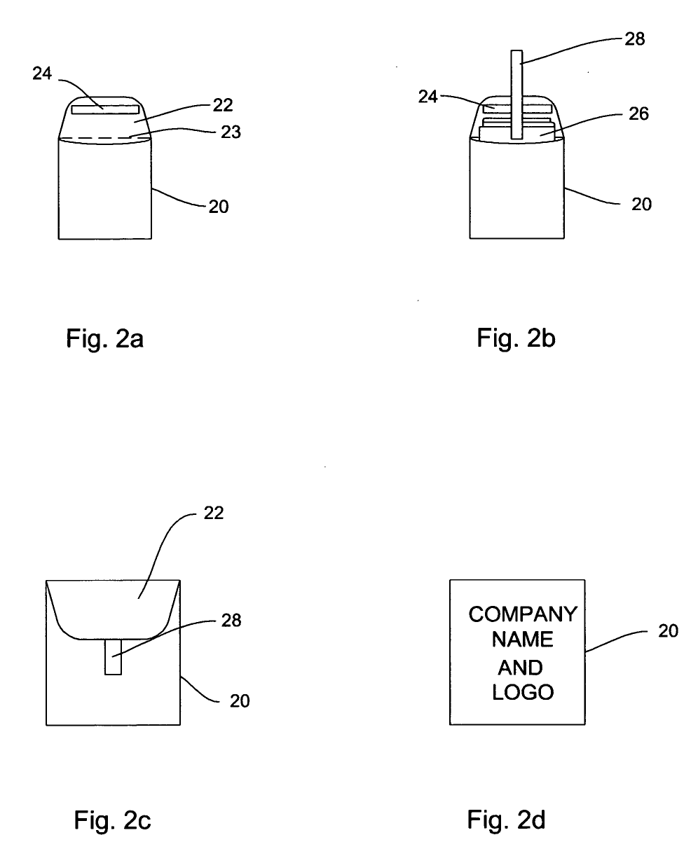

FIG. 2a is a plan view of a backside of the container of FIG. 1 open and without a tissue assembly. The container 20 is constructed similar to a small envelope, bag or pouch and can be made from materials such as paper, paperboard, cloth, or plastic. A flexible plastic film material such as made of polyethylene is presently preferred. In an embodiment the polyethylene film thickness can be somewhere between 1.5 and 2.0 mils (0.0015″-0.002″). The lid 22 is shown in an upward and open position, with fold line 23. A possible adhesive placement area 24 is also shown. A presently preferred adhesive is one that will be permanently attached to the lid, but is resealable to the container to allow repositioning of the lid, for closure and re-closure. The adhesive(s) may be applied by way of a carrier film such as a double-sided tape having a high tack permanent side and a low tack removable (repositionable) side. 3M No. 9415PC (Post-it® type tape) manufactured by 3M (St. Paul, Minn.) is such a tape, and may be used in an embodiment. The tape may also serve the purpose of stiffening and adding strength to the lid, thereby enhancing the performance of a member to lift the lid during dispensing, and prevent stretching or tearing of the thin and flexible lid material.

FIG. 2b is a perspective view of a backside of the container of FIG. 2a open and with a tissue assembly partially inserted. Shown are a container 20, a tissue 26, and a member 28 attached to and protruding from the tissue 26. The tissue assembly consists of the tissue 26 and the member 28. The tissue 26 has been folded to a smaller size before being placed in the container 20.

FIG. 2c is a plan view of a backside of the container of FIG. 2b with a tissue assembly fully inserted and lid 22 closed. The tissue 26 is enclosed in the container 20. One end of the member 28 is attached to the enclosed tissue 26, while another end of the member 28 is exposed to the outside of the container 20. The member 28 had been folded down over the edge of the containers opening, prior to (or during) closure of the lid 22.

FIG. 2d is a plan view of a front side of the container 20 of FIGS. 2a-c on which surface company names and logos can be printed or placed. The container 20 is thus able to serve as a medium for advertising, as well as a means of storage for a tissue.

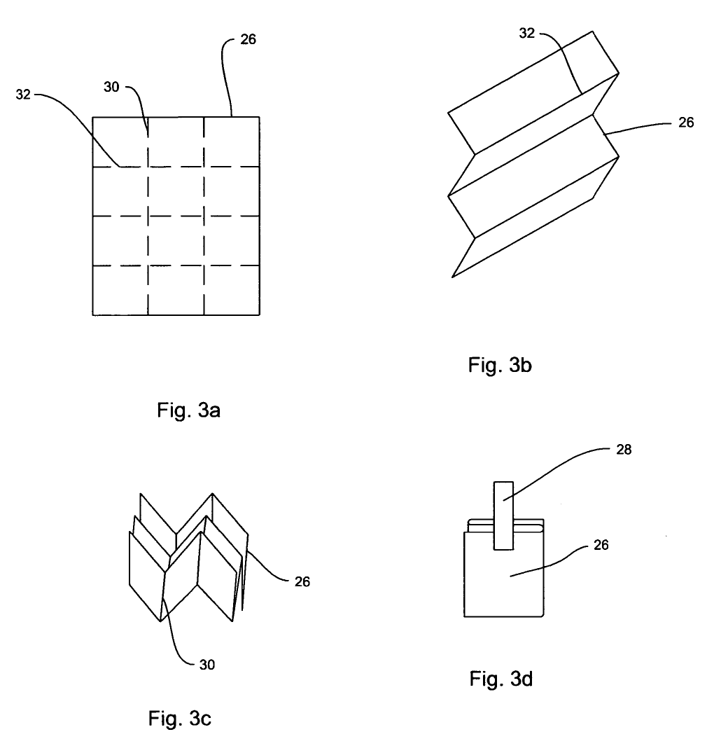

FIGS. 3a-d describes one possible method of constructing a tissue assembly for use in this invention. This is the presently preferred method, as the tissue unfolds rather easily upon being dispensed by the member.

FIG. 3a is a plan view of the tissue 26 shown in FIG. 2b prior to the folding of the tissue 26. Shown are multiple vertical fold lines 30 and multiple horizontal fold lines 32. The fold lines are shown parallel and perpendicular to the edges of the tissue 26. One or more folds in each direction (vertical and horizontal), is anticipated. Additionally, equal or unequal fold line spacing may be used. In an embodiment, an 11″ by 7.5″ tissue can have fold lines spaced so as to make 4 segments by 3 segments, as shown in FIG. 3a, so that each segment is 2.75″ by 2.5″ in size. In an embodiment, a Wypall® L30 wiper manufactured by Kimberly-Clark Corporation (Neenah, Wis.) may be used.

FIG. 3b is a perspective view of the tissue 26 of FIG. 3a depicting a typical first folding of the tissue 26 along horizontal fold lines 32. Fewer, or more, fold lines than shown may be used. A back and forth (zigzag), or wave type of pattern has been used (shown) when folding the tissue 26 along the horizontal fold lines 32. This wave pattern is produced along the vertical sides and any vertical cross-section of the tissue 26, when the tissue 26 is bent along the horizontal fold lines 32 in the manner shown.

FIG. 3c is a perspective view of the tissue 26 of FIG. 3b depicting a typical secondary folding of the tissue 26 along vertical fold lines 30, after the folds depicted in FIG. 3b have been more fully compressed. Fewer, or more, fold lines than shown may be used. A back and forth (zigzag), or wave type of pattern is now similarly produced (to FIG. 3b) in the horizontal direction, as the tissue 26 is now bent along the vertical fold lines 30.

FIG. 3d is a perspective view of the tissue 26 of FIG. 3c further compressed along both horizontal and vertical fold lines to where the segments of the tissue lay relatively flat against one another. Shown is a typical placement of a member 28 to the tissue 26, and where the member 28 is attached at one end. The attachment may be made by various means, such as an adhesive, a stitch, a fastener, or by other means. An adhesive is the presently preferred means of attachment. In an embodiment, a piece of Scotch® permanent double sided tape, manufactured by 3M of St. Paul, Minn. may be used as an adhesive. For a multi-ply tissue, such as a 2-ply, the attachment of the member 28 to the tissue 26 may be placed at a seam-line that holds the different plys together. FIG. 3d depicts a typical and presently preferred folded tissue assembly, comprising a fully folded tissue 26 and a member 28 attached to it by an adhesive. The member 28 may be made of a material that is similar to that of the tissue 26, but which also contains polymer fibers for added pull strength. In an embodiment, a Wypall® X60 wiper manufactured by Kimberly-Clark Corporation (Neenah, Wis.) may be used for a member material. The Wypall® X60 wiper contains both polypropylene fibers and pulp, and may be cut to a short ribbon-like shape. In the description of FIG. 3a above, an embodiment is described where an 11″ by 7.5″ tissue is folded into 2.75″ by 2.5″ segments. In this case, the fully folded tissue shown here in FIG. 3d would be approximately 2.75″ (height) by 2.5″ (width).

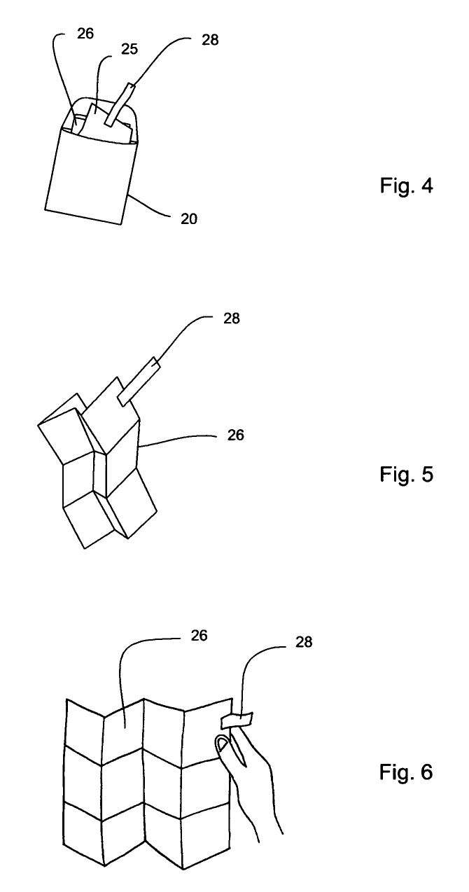

FIGS. 4-9 describes the operation of the presently preferred embodiment, as shown in FIGS. 1 and 2c, beginning with dispensing.

FIG. 4 is a perspective view of the assembly of FIGS. 1 and 2c depicting the dispensing of the enclosed tissue assembly consisting of tissue 26 and member 28, where the tissue 26 may begin to unfold as it is being removed from the container 20. The upper edge and corner 25 of tissue 26 is shown raised from the others. The container 20 for the embodiment described above in the descriptions of FIGS. 3a and 3d (for an 11″ by 7.5″ tissue), may be sized 2.8″ wide, 3″ in height (lid closed), with a 1.3″ long lid 22. This container size should give adequate room for the embodiment of an 11″ by 7.5″ tissue to be easily placed within, and easily removed from the container without having too small of a container for insertion/re-insertion of the tissue, or too large of a container for the palm of a hand.

FIG. 5 is a perspective view of the tissue assembly of FIG. 4 after the tissue assembly has been dispensed and tissue 26 unfolds further.

The unfolding of the tissue 26 occurs as an expansion of the horizontal and vertical “wave forms” that were earlier formed and compressed (during the folding steps FIG. 3a-d). A shake of the hand holding the tissue 26 can further increase this expansion, or rate of expansion, should the tissue 26 not unfold sufficiently on its own.

FIG. 6 is a perspective view of the tissue assembly shown in FIG. 5 unfolded further. FIG. 6 also illustrates a typical hand position taken after the tissue 26 has been removed from the container 20 (shown in FIG. 4) using the member 28, and the hand grasps the nearest edge of tissue 26. Notice that this hand position is one that is most typically desired for tissue use, and is presently preferred for this invention.

A presently preferred embodiment of the container 20 is one that is constructed of such materials and design so as to provide one with a comfortable feeling for allowing the container 20 to remain in ones hand while the tissue 26 is being used. One such material may be a thin flexible plastic film such as made of polyethylene, as was previously mentioned.

FIG. 7 is a perspective view of the empty container 20 of FIG. 4 (and as shown in FIG. 2a), being held with the thumb and forefinger of the hand used to hold the container 20 during dispensing. The lid 22 provides a finger and thumb hold for starting tissue re-insertion. The more randomly compressed (balled-up) used tissue 26 (not shown) can be re-inserted back into the container 20 fairly easily by using a downward curved motion towards the lower inside of the lid 22, as shown by the curved arrow.

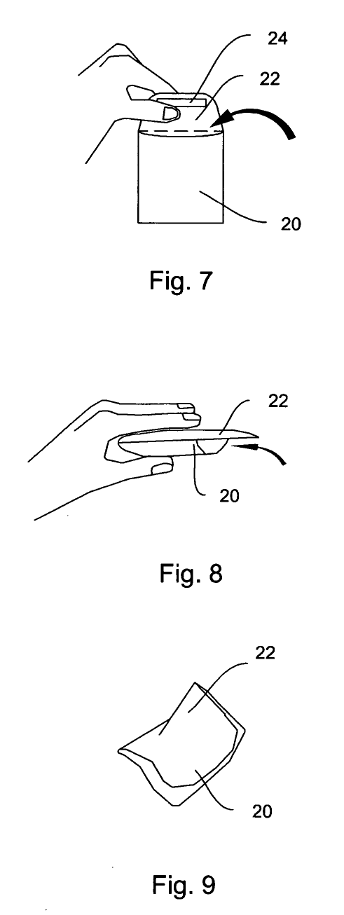

FIG. 8 is a perspective view of another hand position that can be used for re-inserting the tissue 26 into the container 20, the container 20 being shown in a side view. A slightly curved motion similar to that used in FIG. 7 may be used to facilitate re-insertion, again using the lower inside of the lid 22 to deflect the tissue 26 inward. Fingers are shown supporting the lid side of the container 20, while the thumb supports the opposite side.

FIG. 9 is a perspective view depicting a re-packaged tissue assembly after use. The container 20 is much more rounded out, and the adhesive 24 on the inside of the lid 22 (as shown in FIGS. 2a, 2b and 7) allows for re-sealing of the container 20. The container 20 now provides for containment of material on the tissue 26 from passing to the outside environment, and thus provides for better hygiene in applications such as facial tissues, cleaning wipes and medical wipes. This re-confinement of a soiled tissue is especially useful in public areas where immediate disposal means may not be readily available. Quick re-confinement (as well as dispensing) may also be important when dealing with the clean up of unsanitary or hazardous materials, such as might be found in industrial or medical applications.

A description of a typical operation of this presently preferred embodiment of the invention may include the following steps:

a.) Hold the container in one hand with the member facing you, and with the lid located at the top end of the container;

b.) Dispense the tissue by pulling the member upward toward the top end of the container, thereby opening the container and removing the tissue;

c.) Grasp the nearest edge of the tissue by the member, and if necessary, shake the tissue using a downward and upward stroke of the hand (Or, shake with the member and then grab the nearest edge of the tissue);

d.) Use tissue (The container at this time would preferably remain held in the same hand as in step a. above.);

e.) Grasp the lid of the container with the thumb and forefinger of the hand holding it, and reinsert the soiled tissue (One may use a secondary holding position of the container after re-insertion has begun as well, for a two step insertion process; or just use the secondary method. A secondary method is shown in FIG. 8.);

f.) Close and re-seal container; and

g.) Place the used article of the present invention back in one's pocket, or other area desired, until a more proper means of disposal becomes available.

Notice the present invention allows for easier dispensing and more immediate use of a tissue than currently available products. The tissue can be removed directly without first opening the container as a separate step. In the embodiment shown, the tissue comes out fully open (or fully opened with a slight shake of the hand), and with the hand positioned at or near to that desired for use. Also, in the shown embodiment, the container allows for easy re-insertion of the soiled tissue (1 item again). Thus, the soiled tissue is not directly placed into a pocket, purse or bag, or on something, but rather, it's confined and re-packaged so as not to wet or soil something else. All is done easily with two hands, without placing a container down as is most often done with multi-packs of tissues. One hand could easily hold the container of the present invention throughout the process of dispensing, tissue use and re-storage.

Alternate embodiments of a member may include a loop of material where both ends of the material are attached to the tissue; or may include a rigid, semi-rigid, multi-component, or multi-compositional member, such as a rigid component with a flexible seal.

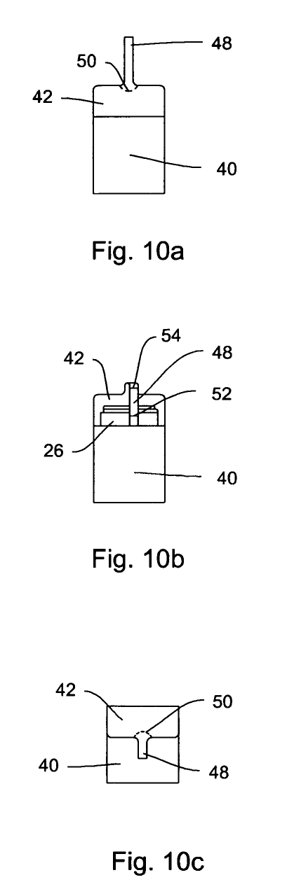

An alternate embodiment of the present invention may use a single piece member-container, as shown in FIGS. 10a-c.

FIG. 10a is a plan view of a member-container 40 whereby a member portion 48 of the member-container 40 may be separated from the member-container 40 by a weakened area 50 such as perforations, as is shown. One end of the member portion 48 is shown attached to a lid 42.

FIG. 10b is a perspective view of the member-container 40 of FIG. 10a depicting a second and opposite end of the member portion 48 attached to a folded and partially inserted tissue 26. Typical fold lines 52 and 54 are also shown, and will be created as the member portion 48 bends and reforms during closure of the lid 42.

FIG. 10c is a plan view of the sealed member-container 40 with folded tissue 26 enclosed, and a section of the member portion 48 exposed. When the member portion 48 is pulled in an upward direction toward the opposite end of the lid 42 from the member portion 48, the member-container 40 opens and the member portion 48 separates from the lid 42 by separating along the weakened area 50.

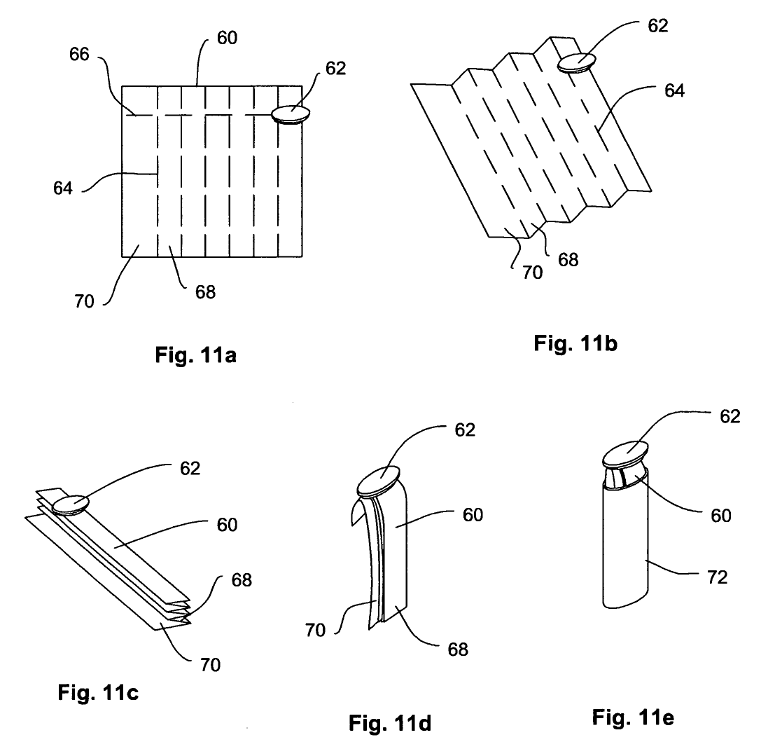

An alternate embodiment of the present invention that may be useful for moist wipe and wet wipe applications is shown in FIGS. 11a-e. A wipe, a method of folding the wipe, and alternate embodiments of a member and container are shown.

FIG. 11a is a perspective view of a wipe assembly comprised of a wipe 60 and attached member 62. The member 62 can be made of an injection molded plastic or elastomer material. The member 62 may also be comprised of more than one component, such as an injection molded plastic with rubber seal. The member 62 could be attached anywhere on the wipe 60, however a location near a corner or edge is preferred. The wipe 60 has multiple vertical fold lines 64, and a single horizontal fold line 66. The multiple vertical fold lines 64 have created multiple sections 68 of equal (or near equal) area with one exception, section 70 which is larger and furthest from the member 62.

FIG. 11b is a perspective view of the wipe assembly of FIG. 11a showing initial folding at the multiple fold lines 64.

FIG. 11c is a perspective view of the wipe assembly of FIG. 11b showing a more fully compressed folded wipe 60 along multiple fold lines 64. Member 62 is shown on top of the folded wipe 60.

FIG. 11d is a perspective view of the wipe assembly of FIG. 11c showing the wipe 60 folded at fold line 66 (shown in FIG. 11a), with the member 62 on the outside of the fold. A portion of section 70 protrudes from the other sections 68 to expose an area of wipe 60 that is furthermost from member 62. By pulling the area of section 70 that is furthermost from the member 62 in a direction away from the member 62, the wipe 60 may be easily unfolded.

FIG. 11e is a perspective view of the wipe assembly of FIG. 11d partially inserted in a container 72. The wipe assembly can be fully inserted so that member 62 comes in contact with and seals to the container 72. Container 72 may be made of an injection molded plastic material.

The example provided by FIGS. 11a-e also demonstrates that by having at least one fold in a different direction from the other folds, a smaller container size can be used.

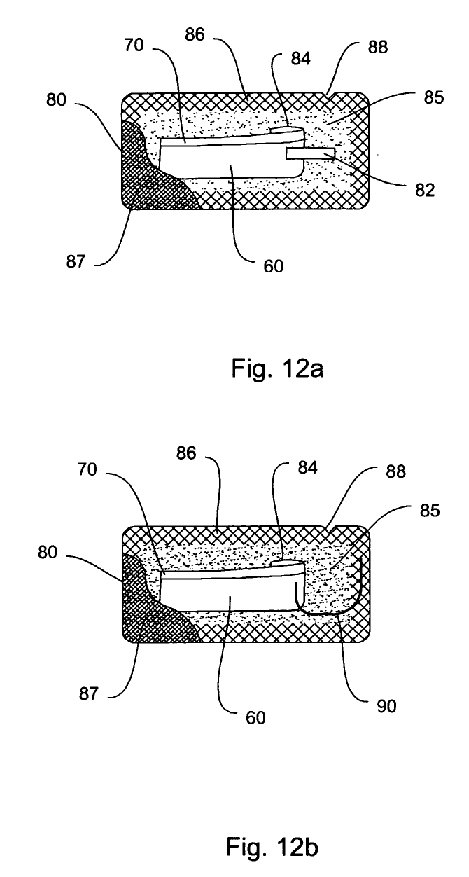

Additional alternate embodiments of the present invention that may be particularly useful for moist and wet wipe applications are shown in FIGS. 12a and 12b. The construction of FIGS. 12a and 12b are similar except for the member. In FIG. 12a the member is totally within the internal cavity of the container. In FIG. 12b, one end of the member is imbedded in the container construction, as is shown in the seal area of the sandwiched packaging films. Current methods of sealing flexible film packaging material may be used. Some methods replace one sealed edge with a folded edge, which is also acceptable.

FIG. 12a is a plan view of the frontal view of the wipe 60, folded as shown in FIGS. 11a-d. A thin flexible rectangular member 82 is shown attached to the wipe 60, in a similar location as is shown in FIG. 11d, or just slightly further down along the longer end of the wipe 60 from the single top fold (FIG. 11d). Two sheets of a flexible packaging film 85 and 87 (cut-away view) are shown sealed along their perimeters. A single sheet of packaging film with a single fold may also be used, with the other three sides sealed. Packaging film, such as a composite of plastic film(s) and metal film that can be heat sealed, may be used. A notch (or cut) 88, is placed in a side of the package as a starting point for developing a preferred placed tear through the package. Once torn open, the wipe assembly consisting of a wipe 60 and member 82 may be removed by use of the member 82. After the wipe assembly has been fully dispensed, pulling member 82 and the exposed wipe area 70 in opposing directions will expand the wipe 60 to a mostly open condition.

FIG. 12b is a plan view of the container 80 and wipe 60 as shown in FIG. 12a, with a member 90 that is attached to the wipe 60 at one end, and is imbedded in the container 80 at it's other end. The imbedded portion of member 90 is shown sandwiched between the two layers of packaging material 85 and 87, within the perimeter seal area 86. When the container 80 is opened by tearing at notch 88 and through the cavity of the container 80 near the seal, the seal area attached to member 90 will become detached from the major portion of the container 80 and accompany the member 90 along with the wipe 60. The detached seal portion can be wrapped or tucked within the wipe 60, prior to the use of wipe 60, to prevent that seal portion from possibly scratching the object to be wiped.

As the above embodiments show, the use of a member greatly enhances the operability of present single moist and wet wipe products, making the dispensing and unfolding of the wipe easy. Likewise, a single tissue product now becomes practical and advantageous, with similar ease of dispensing and unfolding.

While various embodiments of the invention have been disclosed and described herein, it may be apparent to those skilled in the art that various changes in form and detail may be made therein without departing from the spirit and scope thereof.

What is claimed is:

1. An article of sheeted cleaning media for dispensing a single of said sheeted cleaning media, comprising:
a container sized to fit easily within a hand or pocket;
a sheeted cleaning medium; and
a member that is attached to the sheeted cleaning medium and provides a means for removing the sheeted cleaning medium from the container.

2. The article of claim 1 wherein the member provides a means for opening the container.

3. The article of claim 1 wherein the sheeted cleaning medium is a facial tissue, napkin, wet wipe, moist wipe or dry wipe.

4. The article of claim 1 wherein the container is made of polyethylene, or with the use of polyethylene as a material in its construction.

5. The article of claim 1 wherein the container is constructed as an envelope, bag, or pouch, having an attached lid.

6. The article of claim 1 wherein the container has placed upon it an adhesive that provides a means for re-sealing.

7. The article of claim 1 wherein the container lid has placed upon it an adhesive tape that is wholly or partially positioned between the member and the container lid.

8. The article of claim 1 wherein the container is sized appropriately for providing storage space for the sheeted cleaning medium after use, and the container may close and re-seal.

9. The article of claim 1 wherein the sheeted cleaning medium contains chemicals, compounds, or liquids for hygiene, such as moisturizers, medications, surfactants, disinfectants, antibacterial agents, powders, and oils.

10. The article of claim 1 wherein the sheeted cleaning medium is folded back and forth in a wave pattern first in one direction and then again in a second direction, or in one direction and a single fold in a second direction.

11. The article of claim 1 wherein an area of the sheeted cleaning medium is manufactured to be exposed from any other folded sides of the sheeted cleaning medium.

12. The article of claim 1 wherein the member is a short piece of sheeted material, ribbon, or string.

13. The article of claim 1 wherein the member is attached near a corner or edge of the sheeted cleaning medium.

14. The article of claim 1 wherein the member is attached to the sheeted cleaning medium using an adhesive.

15. The article of claim 1 wherein the member is both internally and externally exposed by the container.

16. An article of sheeted cleaning media comprising:
a sheeted cleaning medium; and
a member that is a short piece of sheeted material, ribbon, or string and is attached to the sheeted cleaning medium, to provide a means for dispensing or opening the sheeted cleaning medium.

17. A method for dispensing a sheeted cleaning medium from a container, the steps comprising of:
holding an article of sheeted cleaning media in one hand;
pulling a member that is exposed from the container, or is indirectly exposed from the container by a detachable portion of the container that is attached to the member, with ones other hand, in a direction so as to open the container and dispense the sheeted cleaning medium from the container.

18. The method of claim 17 further including the unfolding of the sheeted cleaning medium to an open, or mostly open condition by shaking.

19. The method of claim 17 further including the unfolding of the sheeted cleaning medium by means of pulling the member and an area of the sheeted cleaning medium that is manufactured to be exposed from any other folded sides of the sheeted cleaning medium in opposing directions.

20. The method of claim 17 further including the reinsertion of a used sheeted cleaning medium to the container, and the container is closed and resealed.
